(19) United States
(12) Patent Application Publication
(10) Pub. No.: US 2007/0282262 A1
(43) Pub. Date: Dec. 6, 2007

(54) Surgical system having integral pneumatic manifolds

(75) Inventors: David L. Williams, Newport Beach, CA (US); Nicolei R. King, Aliso Viejo, CA (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/437,301

(22) Filed: May 19, 2006

(51) Int. Cl. A61M 37/00

(52) U.S. Cl. 604/131

(57) ABSTRACT

A surgical system having all of the various pneumatic control sub-systems integrally mounted on a common manifold. The various required control mechanisms such as valves are likewise integrally mounted to the common manifold.

### BACKGROUND OF THE INVENTION

The present invention relates generally to surgical systems and more specifically to surgical systems that control pneumatic devices.

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worst case, cutting and removal of the retina itself.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port, and the inner member is actuated, closing the port. Upon the closing of the port, cutting surfaces on both the inner and outer cutting members cooperate to cut the vitreous, and the cut vitreous is then aspirated away through the inner cutting member. This cutting action may be made using an electric cutter, but pneumatically driven probes operating at a relatively high pressure are more common.

Additionally, during typical ophthalmic procedures, air may be introduced into the posterior chamber. This air must be of relatively low pressure.

Conventional ophthalmic surgical instrument systems use vacuum to aspirate the surgical site and positive pressure to irrigate the site. Typically, a cassette is serially connected between the means used to generate pressure and the surgical instrument. The use of cassettes with surgical instruments to help manage irrigation and aspiration flows at a surgical site is well known. Aspiration fluid flow rate, vacuum level, irrigation fluid pressure, and irrigation fluid flow rate are some of the parameters that require precise control during ophthalmic surgery. For aspiration instruments, the air pressure is below atmospheric pressure, and fluid is removed from the surgical site. For irrigation instruments, the air pressure is higher than atmospheric pressure, and the fluid will be transported from the irrigation fluid reservoir to the surgical site.

Prior art surgical systems have controlled the various air pressures needed during surgery by using individual manifolds for each sub-system. For example, the aspiration sub-system typically included a manifold having the various required air passages needed to route the vacuum where needed, but the aspiration pump, such as a venturi pump, is mounted remote from the aspiration manifold. This type of construction required that the various subsystems be connected via pneumatic tubing, with the tubing being interrupted at the required valves and “T's”. This sort of construction increases assembly costs, the overall size of the system and can affect reliability.

Accordingly, a need continues to exist for a surgical system having a simplified construction.

### BRIEF SUMMARY OF THE INVENTION

The present invention improves upon prior art by providing a surgical system having all of the various pneumatic control sub-systems integrally mounted on a common manifold. The various required control mechanisms such as valves are likewise integrally mounted to the common manifold.

One objective of the present invention is to provide a surgical system having integrated pneumatic sub-systems.

Another objective of the present invention is to provide a surgical system having pneumatic sub-systems mounted on a common manifold.

Yet another objective of the present invention is to provide a surgical system for controlling pneumatic surgical devices.

These and other advantages and objectives of the present invention will become apparent from the detailed description, drawings and claims that follow.

### BRIEF DESCRIPTION OF THE DRAWINGS

FIG. 1 is a front perspective view of a surgical console that may use the integral pneumatic manifold of the present invention.

FIG. 2 is a front perspective view of a cassette that may be used with the present invention.

FIG. 3 is a rear perspective view of a cassette that may be used with the present invention.

FIG. 4 is an exploded perspective view of the integral pneumatics manifold of the present invention.

FIG. is an enlarged perspective view of the valve or pincher manifold of the present invention and illustrating several active mechanical elements mounted on the manifold.

### DETAILED DESCRIPTION OF THE PREFERRED EMBODIMENTS

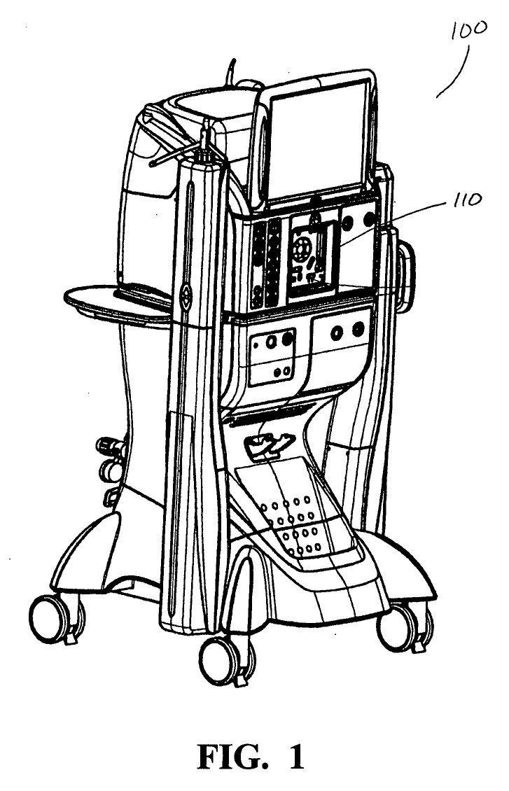

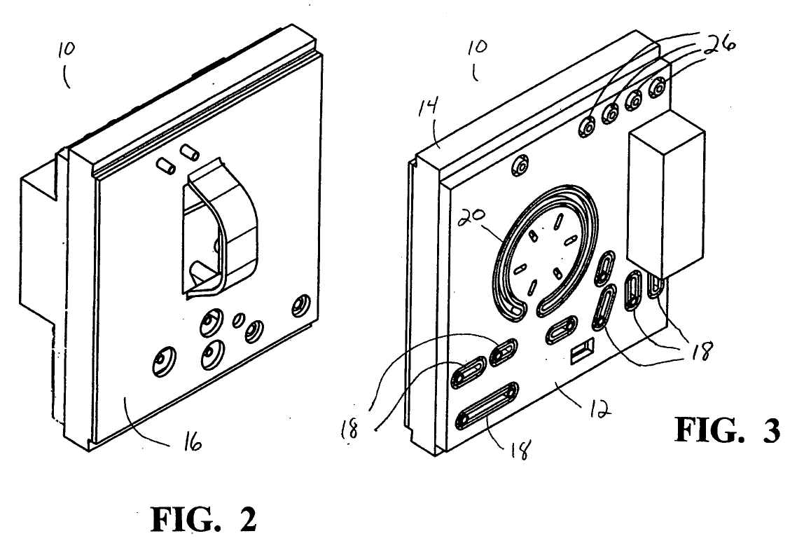

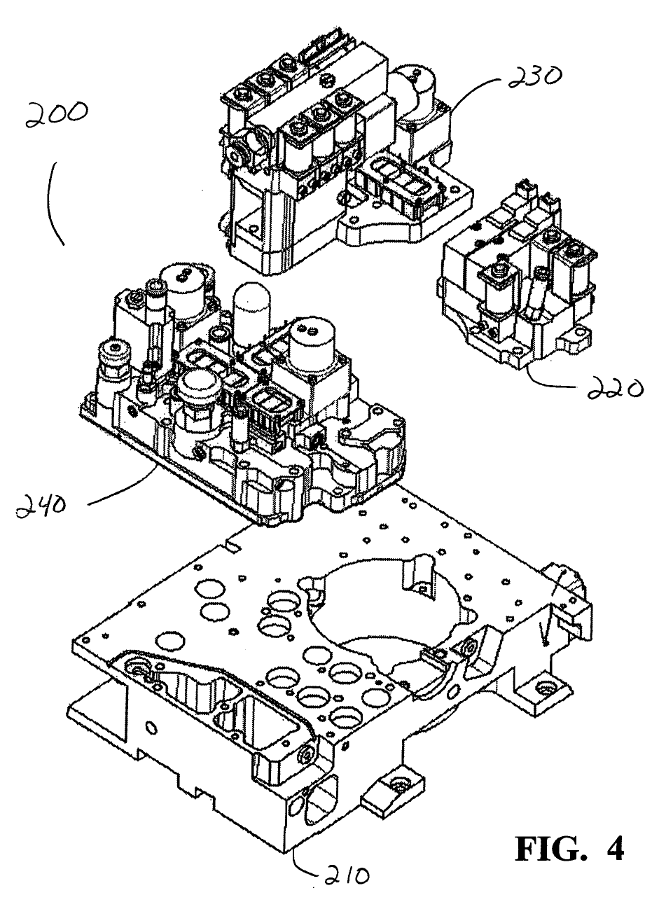

As best seen in FIGS. 1, 2 and 3, cassette 10 that may be used with the present invention generally included valve plate 12, body 14 and cover 16. Valve plate 12, body 14 and cover 16 may all be formed of a suitable, relatively rigid, and thermoplastic. Valve plate 12 contains a plurality of openings 18 and pumping channel 20 that are sealed fluid tight by elastomers 22 and 24, forming a plurality of fluid paths. Ports 26 provide connectors between cassette 10 and surgical console 100 for the various irrigation and aspiration (pneumatic) functions of cassette 10 when cassette 10 is installed in cassette receiving portion 110 of console 100.

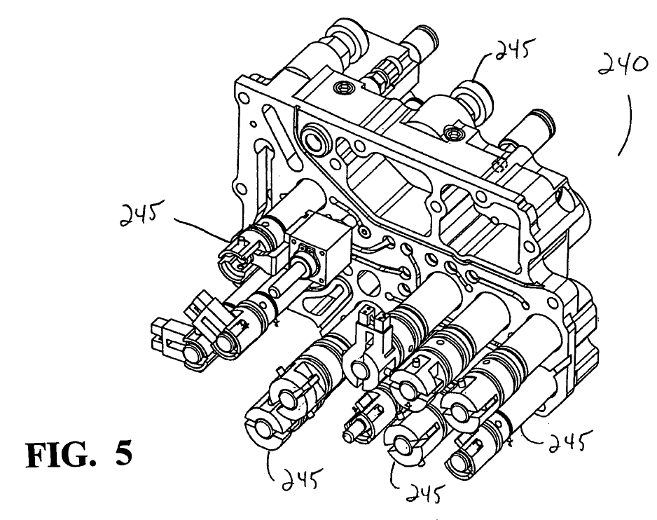

As best seen in FIG. 4, fluidics manifold 200 contains a plurality of sub-assemblies or manifolds mounted to common primary manifold 210. For example, fluidics manifold 200 may additionally contain aspiration manifold 220, and/or infusion/irrigation manifold 230 and/or valve or pincher manifold 240. As seen in FIG. 5, for example, each of manifolds 210, 220, 230 and 240 (manifold 240 used as an illustrative example) are self-contained, and may contain necessary the valves, regulators, sensors or other active embedded mechanical, electrical or electromechanical devices required to perform each manifold's primary function, such as air cylinders 245, by way of example. Manifolds 220 and 230 pneumatically and fluidly communicate with cassette 10 through primary manifold 210, such communication being controlled by pincher manifold 240. Primary manifold 210 may be mounted in cassette receiving portion 110 of console 100 so that cassette 10 may be fluidly coupled to primary manifold 210. Primary manifold 210 may additionally contain pumps and fluid level and/or fluid flow sensors (all not shown).

Such a construction allows for the separation of the primary functionalities of each sub-assembly onto specific manifolds, thereby providing convenient and fast assembly, troubleshooting and repair. In addition, such a construction eliminates the various tubings and tubing connectors used in the prior art to connect the various components in each sub-assembly and reduces the overall size of the completed assembly.

This description is given for purposes of illustration and explanation. It will be apparent to those skilled in the relevant art that modifications may be made to the invention as herein described without departing from its scope or spirit.

We claim:

1. An ophthalmic surgical system, comprising:
a) a primary manifold;
b) an infusion manifold mounted on the primary manifold; and
c) an aspiration manifold mounted on the primary manifold, the aspiration manifold and the infusion manifold pneumatically communicating with each other through the primary manifold.

2. The surgical system of claim 1 further comprising a pincher manifold mounted to the primary manifold and the aspiration manifold and the infusion manifold pneumatically communicate with each other through the pincher manifold.

3. The surgical system of claim 1 wherein the aspiration manifold and the infusion manifold fluidly communicate with each other through the primary manifold.

4. The surgical system of claim 2 wherein the aspiration manifold, the infusion manifold and the pincher manifold fluidly communicate with each other through the primary manifold.

5. The surgical system of claim 2 wherein the aspiration manifold and the infusion manifold fluidly communicate with each other through a cassette.

6. The surgical system of claim 1 further comprising at least one active embedded device mounted on the aspiration manifold and/or the infusion manifold.

7. The surgical system of claim 2 further comprising at least one active embedded device mounted on the aspiration manifold and/or the infusion manifold and/or the pincher manifold

8. The surgical system of claim 2 further comprising at least one active embedded device mounted on the aspiration manifold and/or the infusion manifold and/or the pincher manifold and/or the primary manifold.
